(19) United States
(12) Patent Application Publication
(10) Pub. No.: US 2019/0262793 A1
(43) Pub. Date: Aug. 29, 2019

(54) Polymer-Encapsulated Polyhemoglobin-Based Oxygen Carrier

(71) Applicant: Southwest Research Institute, San Antonio, TX (US)

(72) Inventors: Jian Ling, Spring Branch, TX (US); Rossella Dorati, Villa d'Ogna (BG) (IT)

(21) Appl. No.: 15/907,519

(22) Filed: Feb. 28, 2018

(51) Int. Cl. B01J 13/22; C12N 5/0775; C08J 3/12; B01J 13/10

(52) U.S. Cl. CPC B01J 13/22; C12N 5/0668; C08J 3/126; B01J 13/10; C08J 2471/02; C08J 2389/00; C08J 2465/00; C08J 2467/04; C12N 2500/02

(57) ABSTRACT

PEGylated polyhemoglobin nanocapsules are provided, wherein the nanocapsules each include a core including polyhemoglobin, a polymer shell encapsulating the core, a polydopamine layer on an exterior surface of the shell, and poly(ethylene glycol) adhered by the polydopamine to the shell. A method of forming PEGylated polyhemoglobin nanocapsules includes extracting hemoglobin from red blood cells. The method further includes polymerizing the hemoglobin to provide polyhemoglobin. The polyhemoglobin is then encapsulated in a plurality of polymer shells to form a plurality of polyhemoglobin nanocapsules. In addition, a polydopamine is deposited on an external surface of the polyhemoglobin nanocapsules. Poly(ethylene glycol) is then adhered to the polyhemoglobin nanocapsules with the polydopamine to provide a plurality of PEGylated polyhemoglobin nanocapsules.

### GOVERNMENT SUPPORT CLAUSE

This invention was made with United States Government support under Contract No. W81XWH160035 awarded by the U.S. Army Institute of Surgical Research. The government has certain rights in this invention.

### FIELD

The present disclosure is directed to a polymer-encapsulated polyhemoglobin-based oxygen carrier and, in particular PEGylated polyhemoglobin nanocapsules, and a method of forming thereof.

### BACKGROUND

For the past three decades, or so, oxygen carrier development has been on-going due to civilian and military need of a universal blood substitute, particularly in urgent situations worldwide. Oxygen carriers are expected to replace red blood cells to deliver relatively high concentrations of oxygen to tissue, tissue substitutes, and organs that have a relatively high density of cells under regular metabolism, often at or near normothermic body temperature, e.g., 37° C. Oxygen carriers have broad applications and may be employed in engineered tissue fabrication, ex vivo vascularized tissue and organ perfusion, or serving as a blood substitute in trauma patient resuscitation.

Hemoglobin (Hb) is a tetrameric molecule having a molecular weight of 64 kDa. Hemoglobin is formed of two alpha and beta globin chains, and is the physiological oxygen-transport metalloprotein in red blood cells. However, hemoglobin outside the red blood cell membrane is understood 1) to be toxic to the kidney due to the small molecular size and 2) to cause vasoconstriction and hypertension due to the depletion of nitric oxide (NO) inside the vascular endothelial layers. To address these issues, various formulations of hemoglobin based oxygen carriers (HBOCs) have been developed, mainly divided into two categories: one being polymerized or crosslinked polyhemoglobin and the other being the encapsulation of hemoglobin.

Many polyhemoglobin products performed poorly due to adverse safety issues that arose in Phase II and Phase III clinical trials. The polyhemoglobin with increased molecular size solved the problem of kidney toxicity but did not minimize the side-effect of vasoconstriction and hypertension, as the endothelial layer of blood vessels is still exposed directly to the polyhemoglobin.

Encapsulating hemoglobin in liposomes to mimic red blood cell membranes is another method explored by many researchers. A problem with liposome formulation is that the bilayer may continuously oxidize to methemoglobin, which loses the oxygen carrying capacity. These liposome formulations did not move forward due to the block of antioxidants, such as ascorbic acid, to reduce the oxidation of hemoglobin.

Typical formulations of polymeric nanocapsules used in encapsulation of hemoglobin in co-polymer shells may include biocompatible and biodegradable polymers such as poly(ε-caprolactone) (PCL) or poly(lactic acid) (PLA) in combination with polyethylene glycol (PEG) to form co-polymers. The PEG chains were thought to contribute to the circulation life of the oxygen carrier nanocapsules in the blood or perfusate. It was expected to decrease the surface energy of oxygen carrier nanocapsules and minimize van der Walls attraction and thus reduce nanocapsule aggregation and adsorption of opsonin proteins. The reduction of opsonin protein binding is understood to reduce the chance of being captured by microphages and extend the blood circulation half-life.

However, in testing, the co-polymer formulation demonstrated a shorter than expected circulation life in vivo (tested in rodent) and cytotoxcity in vitro, believed to be due to cellular uptake of the nanocapsules. These problems appear to be related to the ineffectiveness of the PEG chains on the surface of the nanocapsules. In one example, the oxygen carriers with PCL-PEG co-polymer shells were designed to have 5,000 Da molecular weight PEG chains extending outward from the oxygen carrier nanocapsule surface to prevent or reduce the binding of the opsonin proteins. FIG. 1a illustrates a schematic of a PCL-PEG co-polymer nanocapsule exhibiting outwardly projecting PEG chains from a PCL layer with a hemoglobin (Hb) core.

Without being bound to any particular theory, it is believed that in encapsulating the hemoglobin (e.g., using double emulsion), a number of the PEG chains oriented toward the water phase inside the nanocapsule core instead of outside. During fabrication of the polymeric hemoglobin nanocapsules, hemoglobin (or cross-linked polyhemoglobin) dissolved in water is first emulsified and suspended into a solvent, such as ethyl acetate or dichloromethane, that contains the PCL-PEG co-polymer. Some of the hydrophobic PEG chains may orient towards the water phase core containing the hemoglobin (primary water-in-oil emulsion). Subsequently, the primary emulsion further goes through a secondary emulsion (or water-oil-water) process. After the first and secondary emulsion, water phases are present at both the inside and outside of the shells. Some of the PEG chains may change their inward orientation to outward. However, after solvent evaporation and shell hardening, the orientation of the PEG cannot be changed, which may result in an imperfect PEG layer on the nanocapsule surface, such as seen in FIG. lb.

Accordingly, room for improvement remains in the formation of hemoglobin based oxygen carriers and the encapsulation of hemoglobin to provide such oxygen carriers.

### SUMMARY

An aspect of the present disclosure relates to PEGylated polyhemoglobin nanocapsules. The nanocapsules each include a core including polyhemoglobin, a polymer shell encapsulating the core, a polydopamine layer on an exterior surface of the shell, and poly(ethylene glycol) adhered by the polydopamine to the polymer shell. The poly(ethylene glycol) is oriented outwards from the nanocapsules and not present in the core.

It is preferable that the polyhemoglobin exhibits a number average molecular weight (Mn) of 400 kDa to 1,000 kDa. The poly(ethylene glycol) can have a number average molecular weight Mn of 1000-8000. It is also preferable that the poly(ethylene glycol) includes a first poly(ethylene glycol) that exhibits a number average molecular weight (Mn) of 2,000 Da present in the range of 0 to 100 percent by weight and a second poly(ethylene glycol) that exhibits an average molecular weight number (Mn) of 5,000 Da present in the range of 0 percent to 100 percent by weight, wherein the amounts of the polyethylene glycol are selected to achieve 100 percent by weight. It is also preferable that the polymer for the polymer shell is poly(ε-caprolactone). It is further preferable that the PEGylated polyhemoglobin nanocapsule exhibits a size in the range of 70 nm to 250 nm and a polydispersity index in the range of 0.15 to 0.30.

Another aspect of the present disclosure relates to a method of forming PEGylated polyhemoglobin nanocapsules, such as those noted above. The method includes providing polyhemoglobin. The polyhemoglobin is then encapsulated in a plurality of polymer shells to form a plurality of nanocapsules having a core containing polyhemoglobin. In addition, polydopamine is formed on an external surface of the polymer shells (after solvent evaporation and shell hardening) for the polyhemoglobin nanocapsules. Poly(ethylene glycol) is then adhered to the polyhemoglobin nanocapsules with the polydopamine to provide a plurality of PEGylated polyhemoglobin nanocapsules and the poly(ethylene glycol) is oriented outwards from the nanocapsules and not present in the core.

In one embodiment, the method of forming PEGylated polyhemoglobin nanocapsules includes lysing red blood cells and extracting hemoglobin from the red blood cells using a hypotonic lysis buffer and crosslinking the hemoglobin with glutaraldehyde to form polyhemoglobin having a molecular weight in the range of 400 kDa to 1,000 kDa. The polyhemoglobin is then mixed with a first buffer solution having a pH in the range of 6.4 to 6.8 to provide an aqueous phase. Further, a polymer is dissolved in a solvent to form an organic phase. In addition, a first emulsion is formed by adding the aqueous phase dropwise to the organic phase while agitating. Then a second emulsion is formed by mixing the first emulsion with a second buffer solution including a stabilizer and an emulsifier, wherein the second buffer solution has a pH in the range of 8 to 9 to form the nanocapsules. The nanocapsules are then isolated from the second emulsion. A dopamine solution is prepared by adding dopamine to a buffer having a pH in the range of 8 to 9 and the nanocapsules are added to the dopamine solution to coat the nanocapsules with dopamine which forms polydopamine coated polyhemoglobin nanocapsules. The polydopamine coated polyhemoglobin nanocapsules are then added to a poly(ethylene glycol) in solution to form PEGylated polyhemoglobin loaded nanocapsules.

### BRIEF DESCRIPTION OF THE DRAWINGS

The above-mentioned and other features of this disclosure and the manner of attaining them will become more apparent with reference to the following description of embodiments herein taking in conjunction with the accompanying drawings, wherein:

FIG. 1a schematically illustrates a nanocapsule of polyhemoglobin, the nanocapsule including co-polymerized PCL-PEG, wherein the PEG chains are directed out of the nanocapsule and the PCL chains provide a shell to encapsulate the polyhemoglobin core;

FIG. 1b schematically illustrates a nanocapsule of polyhemoglobin, the nanocapsule including co-polymerized PCL-PEG, wherein part of the PEG chains are directed towards the core and part of the PEG chains directed out of the nanocapsule. The PCL chains form a shell to encapsulate the polyhemoglobin core;

FIG. 2 schematically illustrates a method of forming the PEGylated polyhemoglobin nanocapsules;

FIG. 3 schematically illustrates a nanocapsule of polyhemoglobin including a polydopamine coated on the external surface of the nanocapsule shell, and poly(ethylene glycol) adhered to the nanocapsule via the polydopamine;

FIG. 4 is a graph of size and polydispersity index of polyhemoglobin loaded nanoparticles (Poly Hb NPs), polydopamine coated polyhemoglobin loaded nanoparticles (PD Hb NPs), and PEGylated polydopamine coated polyhemoglobin loaded nanoparticles;

FIG. 5 is a graph of Zeta potential of polydopamine coated polyhemoglobin loaded nanoparticles (PD Hb NPs) and PEGylated polydopamine coated polyhemoglobin loaded nanoparticles.

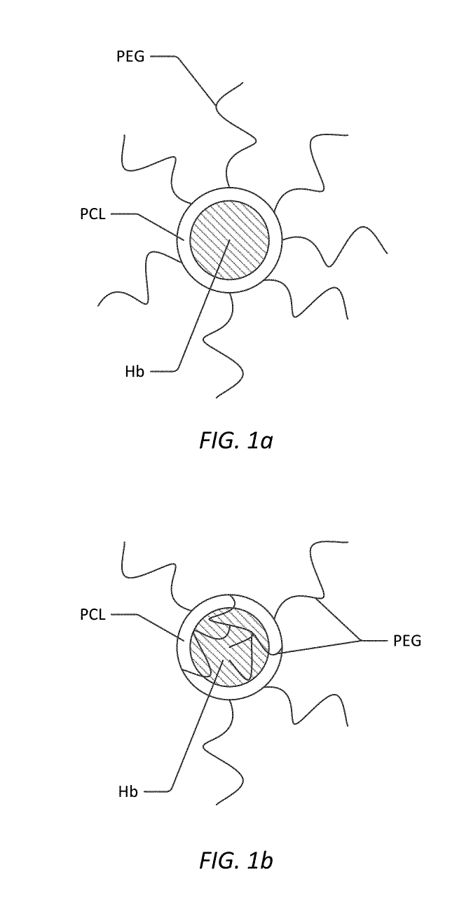

### DETAILED DESCRIPTION

The present disclosure is directed to the encapsulation of polyhemoglobin in a polymer to form a polymer shell. The shell preferably comprises poly(ε-caprolactone) (PCL) surrounding the polyhemoglobin core. Polydopamine is then formed on the outer surface of shell, which preferably comprises depositing of dopamine and the formation of polydopamine. This is followed by affixing, to the preferred polydopamine coating, polyethylene glycol (PEG) polymer chains, which PEG chains do not enter the core. These PEGylated nanocapsules may then provide a polyhemoglobin based oxygen carrier.

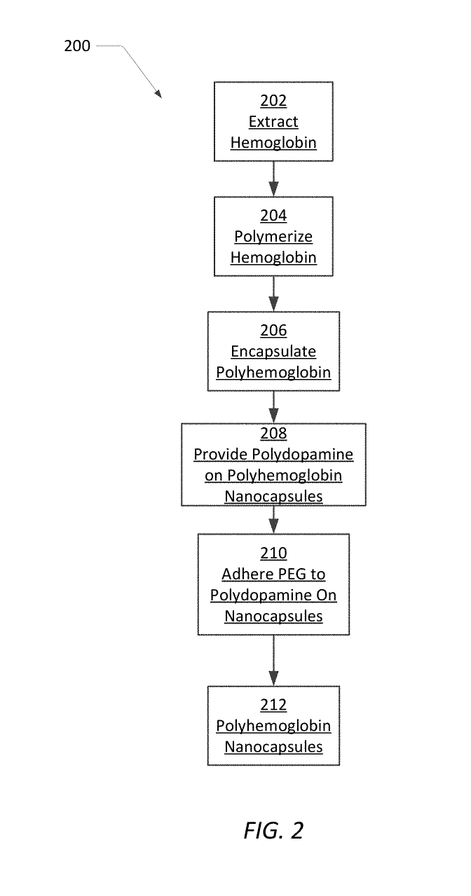

A method of forming the hemoglobin based oxygen carrier (HBOC) nanocapsules 200 is illustrated in FIG. 2 and generally includes extracting hemoglobin from red blood cells 202, polymerizing the hemoglobin to provide polyhemoglobin 204, encapsulating the polyhemoglobin in a plurality of polymer shells to form polyhemoglobin nanocapsules 206, providing polydopamine on an external surface of the polyhemoglobin nanocapsules 208, and affixing poly(ethylene glycol) to the polyhemoglobin nanocapsules with the polydopamine to provide the polyhemoglobin nanocapsules 210. As noted above, the polydopamine is preferably formed by depositing of dopamine which is converted to polydopamine.

Hemoglobin is preferably extracted from animals such as bovine or human red blood cells 202. The cells are preferably lysed by a hypotonic buffer or multiple freeze thaw cycles and cell debris is then removed in multiple filtration steps, including coarse filtration and cross-flow filtration. The hemoglobin may then be stored at 2-8° C., preferably under nitrogen to reduce oxidation. The concentration of the hemoglobin is preferably 6 g/dL_˜8 g/dL. Preferably, the concentration of the hemoglobin is in the range of 1 to 15 g/dL, including all values and ranges therein.

Extracted hemoglobin (Hb) is generally 64 kDa (kilodaltons) in molecular weight. At this weight, however, it was found that the hemoglobin may leak out of the porous polymer shells, particularly when the polymer shells are formed from poly(ε-caprolactone) (PCL). Accordingly, the hemoglobin is preferably polymerized 204 through crosslinking. In embodiments, glutaraldehyde is used as a crosslinking agent to crosslink the hemoglobin to a preferred molecular weight in the range of 400 kDa to 1,000 kDa, including all values and ranges therein.

The polymerized hemoglobin (PolyHb or polyhemoglobin) is then filtered, preferably by cross-flow filtration, to preferably remove over-conjugated polyhemoglobin having a molecular weight of greater than 1,000 kDa as well as non-crosslinked hemoglobin having a molecular weight of less than 100 kDa. The cross-flow filtration process also preferably concentrates the polyhemoglobin prior to encapsulation. In preferred embodiments, the concentration of the polyhemoglobin in solution is in the range of 20 g/dL to 45 g/dL, including all values and ranges therein, such as 28 g/dL. High concentration of PolyHb is found to improve the loading of encapsulation in the next step.

The polyhemoglobin is then encapsulated in an emulsion process 206, and preferably a water-oil-water double emulsion and solvent evaporation process. The core of the nanocapsules is formed by a “water” or aqueous phase. The aqueous phase may be formed by mixing the polyhemoglobin solution obtained after lysis into a first buffer solution including a surfactant. The buffer preferably includes 5 mM TRIZMA buffer exhibiting a pH in the range of 6.4 to 6.8, including all values and ranges therein and preferably 6.6 with 0.5% NaCl. The stabilizer may include poly(vinyl alcohol) (PVA) and is preferably present in an amount in the range of 3.0 to 10.0% w/v (percent weight in the total volume of solution), including all values and ranges therein and preferably 6.0% w/v.

The polymer for forming the polymer shell is prepared in an “oil” or organic phase by dissolving the polymer in a solvent, such as dichloromethane, ethyl acetate or a mixture of dichloromethane and ethyl acetate at a concentration in the range of 1% to 10%, including all values and ranges therein. The polymer is preferably biocompatible and biodegradable. Biocompatible is understood as being not toxic, or injurious, to human tissue and biodegradable is understood as being decomposable by biological means. The polymer preferably includes, e.g., poly(ε-caprolactone) (PCL). Other suitable polymers include poly(lactic-co-glycolic acid) or PLGA as well as poly(lactic acid) or PLA or poly(glycolic acid) or PGA. In embodiments, only a single polymer may be employed; in other embodiments, co-polymers or polymer mixtures may be utilized. An emulsifier, such as Span 80, may also be added to the solvent. The polymer is agitated in the solvent, preferably at room temperature (20° C. to 25° C.), until dissolved.

The aqueous phase providing the core is then preferably added dropwise into the organic phase. The phases form a first emulsion, which is preferably agitated by continuous sonication at 75% amplitude of maximum amplitude or greater, and preferably 90% amplitude of maximum amplitude (depending on the sonication instrument), during the addition of the aqueous phase and maintained in an ice water bath. Addition and sonication lasts for a time period in the range of 1 minute to 10 minutes, including all values and ranges therein and preferably 3 minutes for a 3 to 5 mL of total first emulsion volume (the emulsion time depends on the total emulsion volume). While agitation by sonication is preferred, other forms of agitation (e.g., homogenization) may be utilized instead. This first emulsion provides a water-oil system.

After forming the first emulsion, the first emulsion is poured into a second buffer solution forming a second emulsion of the organic phase. The second buffer is preferably 5 mM TRIZMA buffer, having a pH in the range of 8 to 9, including all values and increments therein and preferably 8.66, with 0.5% NaCl. The second buffer solution may include a surfactant, such as poly(vinyl alcohol) (PVA), present in an amount in the range of 3 to 10% w/v including all values and ranges therein and more preferably 6% w/v. The second buffer solution may also include a surfactant, such as sodium docecyl sulfate (SDS), present in the range of 0.05% w/v to 5.0% w/v including all values and ranges therein and preferably 0.1% w/v. The second emulsion is preferably agitated, such as by sonication, for a period of time in the range of 1 to 5 minutes, including all values and ranges therein and preferably 3 minutes, for a 15 to 25 mL of total second emulsion volume (the emulsion time depends on the total emulsion volume), at an amplitude in the range of 25% to 50% of maximum amplitude (depending on the sonication instrument), including all values and ranges therein, such as 40%, in an ice water bath at a temperature in the range of 2° C. to 8° C., including all values and ranges therein. Again, while sonication is preferred, other forms of agitation (e.g., homogenization) may alternatively be utilized.

Additional buffer solution is preferably added to the second emulsion. After addition of additional buffer solution, the second emulsion is preferably agitated through stirring for 2 to 4 hours, including all values and ranges therein such as 3 hours for solvent evaporation. The organic solvent evaporation time 3 hours is for 3 mL of organic solvent in about 75 mL of buffer solution. The time period of solvent evaporation depends on the volume of organic solvent, the volume of buffer included in the experiment, the temperature, as well as the pressure/vacuum. Typically, the solvent evaporation is performed in an ice bath at a temperature in the range of 2° C. to 8° C., including all values and ranges therein under atmosphere pressure.

After solvent evaporation, the surfactant may then be removed from the resulting polyhemoglobin nanocapsules by centrifugation or cross-flow filtration. The polyhemoglobin nanocapsules are then re-suspended in de-ionized water (DI water) including a surfactant, such as SDS, present in the range of 0.01% w/v to 10% w/v, including all values and ranges therein and preferably 0.07% w/v.

The polyhemoglobin nanocapsules are then coated with a polydopamine 208 by incubating the nanocapsules in dopamine solution. The polydopamine then associates with the PEG therein surrounding the polyhemoglobin nanoparticles where the PEG is now oriented outwards from the nanoparticle PCL surface and not present in the polyhemoglobin core.

Polydopamine (Poly-Dp) is preferably added as dopamine hydrochloride to a solvent, which is preferably a buffer, such as TRIZMA buffer, having a pH in the range of 8.0 to 9.0, including all values and ranges therein and preferably 8.66. The dopamine may be added at 0.01% w/v to 0.05% w/v, including all values and ranges therein, and preferably 0.026% w/v (or 0.26 mg/mL). Polydopamine is then formed from oxidation of the dopamine. Reference to polydopamine herein may be understood as either covalently attached dopamine monomer units via oxidative polymerization or self-assembled dopamine and its oxidative product that may be held together by electronic associations (e.g. charge transfer, 7C stacking or hydrogen bond interactions). The polyhemoglobin nanocapsules are added to the solution containing the polydopamine and the polydopamine coats the external surfaces of the nanocapsules. The mixture is stirred to provide mild agitation for 10 to 14 hours, including all values and ranges therein and preferably 12 hours, in the dark.

Free dopamine and/or polydopamine is then preferably removed by centrifugation or cross-flow filtration The polydopamine coated polyhemoglobin nanocapsules are then re-suspended in a solution of surfactant, preferably SDS, present in the range of 0.01% w/v to 10% w/v, including all values and ranges therein and preferably 0.07% w/v and de-ionized water.

The polydopamine coated nanocapsules are then PEGylated 210. PEGylation is understood herein as the process of affixing poly(ethylene glycol) polymer chains to the polydopamine coating on the nanocapsules. The multiple functional groups presented on polydopamine coatings are able to react with a wide range of molecules. In this case, the oxidized quinone form of catechol can undergo reactions with amine and thiol groups via Michael addition or a Schiff base reaction to form covalently grafted functional layers. In this study, linear PEG at one end terminated with amine group, i.e., amine-PEG (1 ARM) (PEG-NH₂), was used to bind to the polydopamine layer. Other forms of PEG terminated with an amine group or a thiol group also can be used to bind the polydopamine layer. The solvent may again be a buffer solution, preferably TRIZMA buffer, at a pH in the range of 8.0 to 9.0 including all value and ranges therein, such as 8.66. The PEG may be selected based upon its number average molecular weight value or Mn. Preferably, the Mn value is in the range of 1000 Daltons (Da) to 10,000 Da. In addition, preferably, one may select PEG for coating from two or more different Mn values, where the Mn values differ by at least 1000 Da, or by at least 2000 Da, or by at least 3000 Da. For example, one may select PEG for coating form one sample of PEG having a Mn value of 2000 Da and one PEG sample having a Mn value of 5000 Da.

Accordingly, poly(ethylene glycol) of varying molecular weights may be provided at different percentages of the total amount by weight (w/w) of poly(ethylene glycol), such as 10:90 to 90:10 including all values and ranges therein, and preferably in the range of 25:75 to 75:25. In particularly preferred embodiments, the PEG includes a first poly(ethylene glycol) of 2,000 Da number average molecular weight (Mn) provided in the range of 75 percent w/w to 25 percent w/w and a second poly(ethylene glycol) of 5,000 Da Mn provided in the range of 25 percent w/w to 75 percent w/w. It may be appreciated that the Mn number, such as 2,000 Da or 5,000 Da, indicates the number average molecular weight of the poly(ethylene glycol) and not all of the poly(ethylene glycol) chains classified under a particular number average molecular weight are necessarily the same molecular weight.

Besides the choice of mixing ratio among different PEG ratio, the total PEG concentration (milligram in milliliter) used to bind the polydopamine layer can also be optimized, for example, from 1 mg/mL to 100 mg/mL, prefer to be 30 mg/mL.

To the poly(ethylene glycol) solution, the polydopamine coated polyhemoglobin nanocapsules are preferably added. The solution is preferably stirred at room temperature for a period of time in the range of 2 hours to 12 hours, including all values and ranges therein, such as 4 hours, in the dark. Free poly(ethylene glycol) may then be removed by centrifugation or cross-flow filtration.

After centrifugation, the post PEGylated nanocapsules are preferably re-suspended 212 in de-ionized water and a surfactant, such as SDS, wherein the surfactant is present in the range of 0.10%w/v to 0.20% w/v, including all values and ranges therein, such as 0.14% w/v The suspended PEGylated polyhemoglobin nanocapsules may then be stored at temperatures in the range of 0° C. to less than 20° C., preferably under nitrogen, for later use.

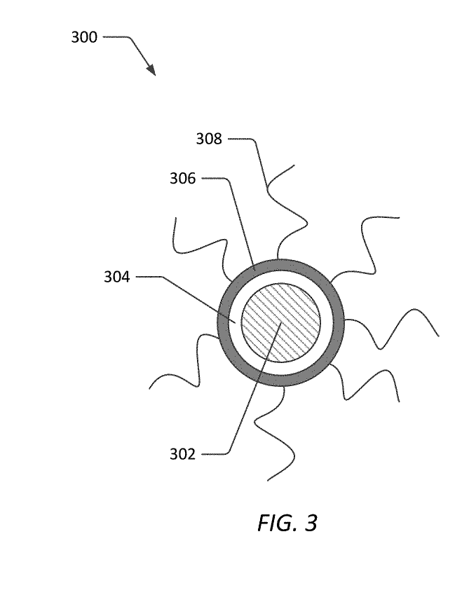

Accordingly, nanoencapsulation of the hemoglobin preferably produces nanocapsules such as the preferred embodiment schematically illustrated in FIG. 3. The illustrated PEGylated polyhemoglobin nanocapsules 300 include polyhemoglobin (Hb) in the core 302 with a layer of polymer, preferably poly(ε-caprolactone) (PCL) encapsulating the core providing a shell 304. A layer of polydopamine (P-Dp) 306 is present over the shell 304 and then PEG 308 is adhered to the shell via the P-Dp to PEGylate the nanocapsules. Reference to the PEG 308 as adhered to the P-Dp is reference to either covalent bonding of the PEG to the P-Dp or electronic association.

As can be seen, the PEG is oriented outwards from the nanocapsule surface. Accordingly, with respect to the nanocapsules herein, ≥95.0 wt. % of the PEG present is oriented outwards from the nanocapsule shell 304, more preferably ≥96.0 wt. %, or ≥97.0 wt. %, or ≥98.0 wt. %, or ≥99.0 wt. %, or even 100 wt. % of the PEG present is oriented outwards from the nanocapsule shell 304 and not present in the nanocapsule core 302 containing the polyhemoglobin.

The PEGylated polyhemoglobin nanocapsules exhibit a size preferably in the range of 70 to 250 nm, including all values and rangers therein. The polydispersity of the PEGylated polyhemoglobin nanocapsules size distribution is preferably to be less than 0.30. The smaller the polydispersity index, the more uniform of the nanoparticles in size. It is noted that PEGylating the polyhemoglobin nanocapsules by affixing the poly(ethylene glycol) with a polydopamine allows for a degree of control over polyethylene glycol chain length, the mixture of different polyethylene glycol chain lengths, and the density of the polyethylene glycol polymer chains on the surface of the nanocapsules. In addition, the method and formulation presently reduces cost over co-polymerized PCL-PEG systems as co-polymerized PCL-PEG is significantly more expensive than the cost of PCL and PEG individually.

### EXAMPLE

### Hemoglobin Extraction

Hemoglobin was extracted from bovine red blood cells after being lysed by a hypotonic buffer. Cell debris was eliminated via multiple filtration processes including coarse filtration followed by cross-flow filtration. A hemoglobin quantification assay, the Drabkin assay, was set up to measure the hemoglobin concentration after extraction, which was determined to be around 7 g/dL in hypotonic 3.75 mM phosphate buffer. The assay was also used to quantify the loading efficiency.

### Polyhemoglobin (PolyHb) Formation

Hemoglobin extracted from red blood cells is 64 kDa in molecular weight. The hemoglobin was polymerized to obtain polyhemoglobin of around 400 kDa to 1,000 kDa via glutaraldehyde-based crosslinking before the encapsulation process. After crosslinking, a cross flow filtration (CFF) process was used to eliminate over-conjugated hemoglobin particles of greater than 1,000 kDa as well as non-cross-linked hemoglobin less than 100 KDa. The CFF process also concentrated the polyhemoglobin for the encapsulation process.

### Nano-encapsulation of PolyHb in PCL Shells

Poly(ε-caprolactone) (PCL) encapsulated polyhemoglobin was prepared using the water-oil-water (w-o-w) double emulsion and solvent evaporation method described below. Generally, the preparation procedure of the polyhemoglobin loaded nanocapsules was performed by a double emulsion and solvent evaporation of technique: (a) first (water-oil) emulsion containing polyhemoglobin in aqueous solution emulsified into PCL dissolved-solvent dichloromethane was prepared by sonication, (b) second (water-oil-water) emulsion was prepared by (a) further emulsified in aqueous solution via sonication, and (c) the “water-oil-water” emulsion after second emulation is ready for solvent evaporation phase.

The encapsulation procedure of this example is described below (this procedure is scalable):
1. Prepare “oil” phase or organic phase by adding 250 mg (8.3% w/v) of Span 80 and 120 mg of PCL powder in a clear vial with cap. Add 3 mL of dichloromethane (DCM) and close the vial. Maintain stirring at room temperature until the polymer is completely dissolved.
2. Prepare 10 mL of PVA (6% w/v) in 5 mM TRIZMA® buffer (pH=6.6) +0.5% NaCl.
3. Prepare 25 mL of PVA (6% w/v) in 5 mM TRIZMA® buffer (pH=8.66) +0.5% NaCl in a 50 mL beaker. Keep stirring at 2-8° C.
4. Prepare 50 mL of PVA (6% w/v) in 5 mM TRIZMA® buffer (pH=8.66) +0.5% NaCl in a 100 mL beaker and keep stirring at 2-8° C.
5. Add 0.1% w/v SDS to each of the two PVA solutions in above 3 and 4.
6. Prepare “water” phase or Internal Aqueous Phase by mixing 1 mL PolyHb solution (at ˜28 g/dL concentration) into 1 mL PVA (6% w/v) in pH=6.6 TRIZMA® buffer +0.5% NaCl. Mix and maintain the solution in ice/water bath.
7. Start the first emulsion by adding 0.5 mL of Internal Aqueous Phase into 3 mL of Organic Phase drop-by-drop using a 1 mL syringe with a 30G needle while maintaining the system under continuous sonication, at 90% amplitude, for 3 minutes in ice/water bath.
8. Pour the (water-oil) sample after first emulsion into the 25 mL of 6% (w/v) PVA solution in TRIZMA® buffer (pH=8.66)+0.5% NaCl, and 0.025 mg SDS (0.1% w/v). Mix the suspension and perform continuous sonication at 40% amplitude for 3 minutes, maintain the system in ice/water bath during the sonication.
9. Pour the suspension above further into the 50 mL of 6% (w/v) PVA solution in TRIZMA® buffer (pH=8.66) +0.5% NaCl and 0.050 g of SDS (0.1% w/v).
10. Maintain the suspension under magnetic stirring (speed #3), under hood, for 3 hours solvent evaporation on ice/water bath.
11. Remove PVA by centrifugation or cross-flow filtration. When using the centrifugation, the sample suspension is centrifuged at 12,000×g (centrifugation force) for 40 min at 20° C.
12. After centrifugation, the sample is re-suspended to 3 mL of 0.07% SDS in Millipore DI water.
   The Coating of Polyhemoglobin Loaded PCL Nanocapsules with Polydopamine (PolyDp)

An example of the polydopamine coating procedure is described below (this procedure is scalable):
1. Prepare 0.26 mg/mL dopamine solution by adding 13 mg of dopamine (dopamine hydrochloride H8502) to 47 mL of TRIZMA buffer (pH=8.66).
2. Add 3 mL of PCL encapsulated polyHb nanocapsules suspension (in 0.07% SDS in Millipore DI water) and maintain the suspension under stirring (mild agitation), at room temperature for 12 hours in the dark.
3. Remove free dopamine and polydopamine by centrifugation the sample at about 155,000×g for 3 hours, at 20° Cg.
4. After centrifugation, re-suspend the pellet in 3 mL of 0.07% SDS in Millipore DI water.
5. Alternatively, the polydopamine coated nanocapsules are isolated using cross-flow filtration.
   The Coating of Polyhemoglobin Loaded PCL Nanocapsules with Poly(Ethylene Glycol) (PEG)
1. Combine 7 mL of TRIZMA buffer (pH 8.66), 25 mg PEG-NH₂ 2000 Da (1-ARM) and 75 mg PEG-NH₂ 5000 Da (1-ARM) in a vial (The PEG 2000 Da and PEG 5000 Da ratio can be 25:75, or 50:50, or 75:25 w/w), and stir until fully dissolved.
2. Add 3 mL re-suspended nanocapsules after polydopamine coating.
3. Maintain under magnetic stirring (speed #3), at room temperature, for 4 hours, in the dark.
4. After 4 hours, remove free PEGs by centrifuging the sample at 155,000×g for 45 minutes, at 20° C..
5. After centrifugation, re-suspend the pellet in 1 mL of 0.14% SDS in Millipore DI water.
6. Store nanocapsules in the refrigerator for further use.
7. Alternatively, the PEGylated nanocapsules are isolated using cross-flow filtration.

### Sizes and Stability of Nanocapsules at Different Stages

The average nanocapsule size, polydispersity index (PI), and Zeta potential (representing stability) were measured after each processing stage: 1) PCL encapsulation of polyhemoglobin, 2) coating the polyhemoglobin nanocapsules with polydopamine, and 3) coating the polydopamine coated polyhemoglobin nanocapsules with a mixture of PEG 2000 Da Mn and PEG 5000 Da Mn. Zeta potential of polydopamine coated nanocapsules was measured at time zero (immediately after the coating steps).

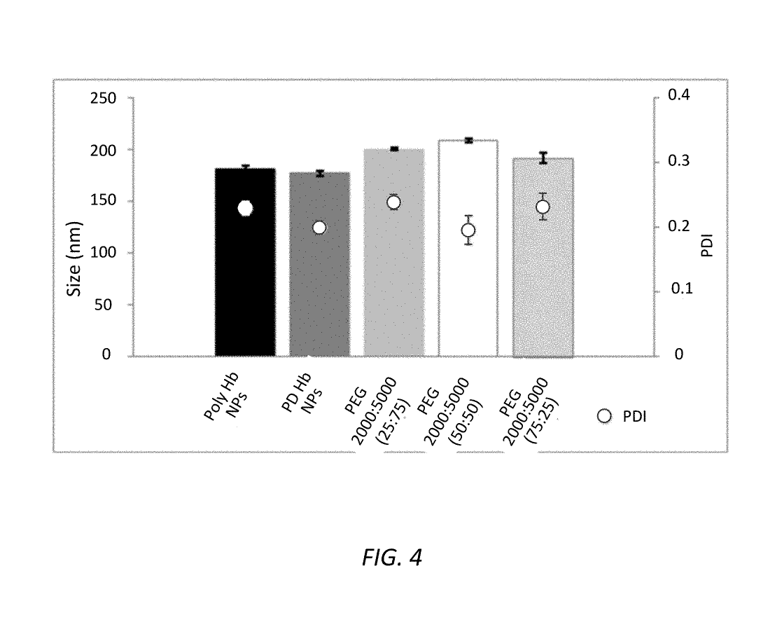

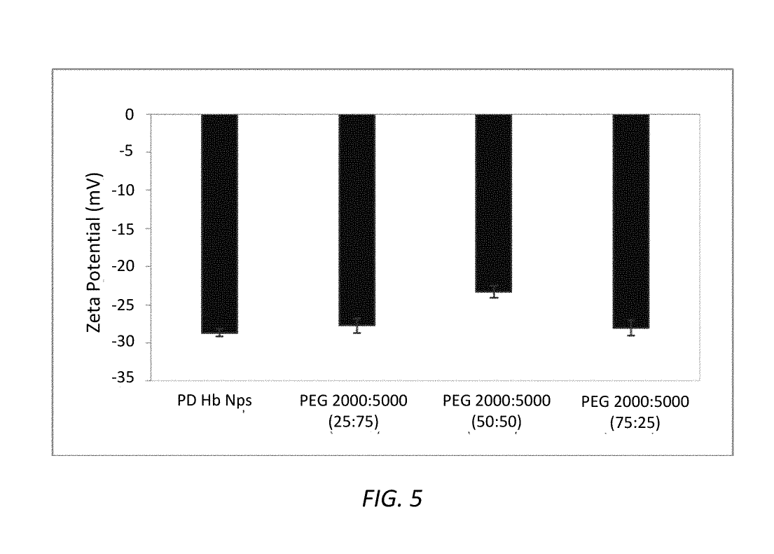

The measurements are listed in Table 1, as well as illustrated in FIGS. 4 and 5. The data indicates that PEGylation leads to a slight increase of nanocapsule size. The PEG 2000:5000 ratio at both 25:75 and 75:25 w/w had the largest Zeta potential absolute values (or most stable).

TABLE 1
Characterization

| | Particle Size | | Zeta potential |
|---|---|---|---|
| Description | (nm) | PI | (mV) |
| PCL-PolyHb^{a} | 182 ± 2.4 | 0.23 ± 0.01 | −6.77 ± 0.49 |
| PCL-PolyHb-PolyDp^{b} | 177.3 ± 2.9 | 0.20 ± 0.01 | −28.7 ± 0.96 |
| PCL-PolyHb-PolyDp-PEG^{c} | 200.6 ± 1.2 | 0.24 ± 0.01 | −27.7 ± 0.79 |
| PEG2000:5000 (25:75) | | | |
| PCL-PolyHb-PolyDp-PEG^{c} | 209.3 ± 1.6 | 0.20 ± 0.02 | −23.4 ± 1.00 |
| PEG2000:5000 (50:50) | | | |
| PCL-PolyHb-PolyDp-PEG^{c} | 192.5 ± 5.4 | 0.23 ± 0.02 | −28.06 ± 0.53 |
| PEG2000:5000 (75:25) | | | |

^{a}PCL encapsulated polyhemoglobin nanocapsules;
^{b}PCL encapsulated polyhemoglobin nanocapsules coated with polydopamine on surfaces;
^{c}Post-PEGylated PCL encapsulated polyhemoglobin nanocapsules with polydopamine surface coating.

FIG. 4 illustrates the size and polydispersity values of polyhemoglobin loaded nanocapsules (PolyHb Nps), polydopamine coated polyhemoglobin nanocapsules (DP-PCL-PolyHb Nps) and PEG coated polyhemoglobin nanocapsules. FIG. 5 illustrates the Zeta potential of the polyhemoglobin nanocapsules after coating with various Mn mixtures of PEG-NH₂ 2000 Da and PEG-NH₂ 5000 Da at different ratio (25:75, 50:50 and 75:25 w/w).

1. A method of forming PEGylated polyhemoglobin nanocapsules, comprising:
supplying polyhemoglobin;
encapsulating said polyhemoglobin in a plurality of polymer shells to form a plurality of nanocapsules having a core containing polyhemoglobin;
providing a polydopamine coating on said polymer shell; and
adhering poly(ethylene glycol) to said polyhemoglobin nanocapsules with said polydopamine to provide a plurality of PEGylated polyhemoglobin nanocapsules wherein said poly(ethylene glycol) is oriented outwards from said nanocapsules.

2. The method of claim 1, wherein said polyhemoglobin is formed from hemoglobin extracted from bovine or human red blood cells.

3. The method of claim 1, wherein said polyhemoglobin is form from hemoglobin extracted from red blood cells by lysing said red blood cells using a hypotonic lysis buffer.

4. The method of claim 1, wherein said polyhemoglobin is formed by polymerizing hemoglobin with glutaraldehyde.

5. The method of claim 1, wherein said polyhemoglobin exhibits a number average molecular weight (Mn) in the range of 400 kDa to 1,000 kDa.

6. The method of claim 1, wherein encapsulating said polymerized hemoglobin in said polymer shells comprises:
mixing said polyhemoglobin with a first buffer solution having a pH in the range of 6.4 to 6.8 to provide an aqueous phase;
dissolving a polymer in a solvent to form an organic phase;
forming a first emulsion by adding said aqueous phase dropwise to said organic phase while agitating;
mixing said first emulsion with a second buffer solution including a stabilizer and an emulsifier or surfactant, wherein said second buffer solution has a pH in the range of 8 to 9, to form a second emulsion and forming said polyhemoglobin nanocapsules; and
isolating said polyhemoglobin nanocapsules from said second emulsion.

7. The method of claim 1, wherein said polymer shells are formed from poly(ε-caprolactone) (PCL), poly(lactic acid) (PLA), poly(lactic-co-glycolic acid) (PLGA) or poly(glycolic) (PGA).

8. The method of claim 1, wherein said poly(ethylene glycol) includes poly(ethylene glycol) exhibiting a number average molecular weight (Mn) of 2,000 Da present in the range of 75 to 25 percent of the total weight of poly(ethylene glycol) and poly(ethylene glycol) exhibiting a number average molecular weight (Mn) of 5,000 Da present in the range of 25 percent to 75 percent of the total weight of the poly(ethylene glycol), wherein said amounts of poly(ethylene glycol) are selected to achieve 100% of the total weight.

9. A method of forming PEGylated polyhemoglobin nanocapsules, comprising:
lysing red blood cells and extracting hemoglobin from said red blood cells using a hypotonic lysis buffer;
crosslinking said hemoglobin with glutaraldehyde to form polyhemoglobin having a number average molecular weight in the range of 400 kDa to 1,000 kDa;
mixing said polyhemoglobin with a first buffer solution having a pH in the range of 6.4 to 6.8 to provide an aqueous phase;
dissolving a polymer in a solvent to form an organic phase;
forming a first emulsion by adding said aqueous phase dropwise to said organic phase while agitating;
forming a second emulsion by mixing said first emulsion with a second buffer solution including a stabilizer and an emulsifier, wherein said second buffer solution has a pH in the range of 8 to 9 to form said nanocapsules;
isolating said nanocapsules from said second emulsion;
preparing a dopamine solution by adding dopamine to a buffer having a pH in the range of 8 to 9;
adding said nanocapsules to said dopamine solution and coating said nanocapsules with said dopamine to form polydopamine coated polyhemoglobin nanocapsules; and
isolating the polydopamine coated polyhemoglobin nanocapsules, and
adding said polydopamine coated polyhemoglobin nanocapsules to a poly(ethylene glycol) in solution to form PEGylated polyhemoglobin loaded nanocapsules.

10. The method of claim 9, wherein said red cells are from a bovine or a human.

11. The method of claim 9, wherein said polymer is poly(ε-caprolactone) (PCL), or poly(lactic acid) (PLA), or poly(lactic-co-glycolic acid) (PLGA) or poly(glycolic) (PGA).

12. The method of claim 9, wherein said poly(ethylene glycol) includes poly(ethylene glycol) exhibiting a number average molecular weight (Mn) of 2,000 Da present in the range of 75 to 25 percent by weight and poly(ethylene glycol) exhibiting a number average molecular weight (Mn) of 5,000 Da present in the range of 25 percent to 75 percent by total weight, wherein said amounts of poly(ethylene glycol) are selected to achieve 100% of the total weight.

13. A plurality of PEGylated polyhemoglobin nanocapsules, said nanocapsules each comprising:
a core including polyhemoglobin;
a polymer shell encapsulating said core having an exterior surface;
a polydopamine layer on said exterior surface of said polymer shell; and
poly(ethylene glycol) adhered by said polydopamine to said polymer shell.

14. The PEGylated polyhemoglobin nanocapsule of claim 13, wherein said polyhemoglobin exhibits a number average molecular weight (Mn) of 400 kDa to 1,000 kDa.

15. The PEGylated polyhemoglobin nanocapsule of claim 13, wherein said poly(ethylene glycol) includes a first poly(ethylene glycol) exhibiting a number average molecular weight (Mn) of 2,000 Da present in the range of 75 to 25 percent by weight and a second poly(ethylene glycol) exhibiting a number average molecular weight number (Mn) of 5,000 Da present in the range of 25 percent to 75 percent by weight, wherein said amounts of said polyethylene glycol are selected to achieve 100 percent by weight.

16. The PEGylated polyhemoglobin nanocapsule of claim 13, wherein said polymer is poly(ε-caprolactone).

17. The PEGylated polyhemoglobin nanocapsule of claim 13, wherein said PEGylated polyhemoglobin nanocapsule exhibits a size in the range of 70 nm to 250 nm and a polydispersity index in the range of 0.15 to 0.30.

18. A method of increasing human mesenchymal cell metabolism, comprising:
adding PEGylated polyhemoglobin nanocapsules to human mesenchymal cells in a cell culture medium, wherein said PEGylated polyhemoglobin nanocapsules comprise a core including polyhemoglobin, a polymer shell encapsulating said core, a polydopamine layer coating on an exterior surface of said polymer shell, and polyethylene glycol adhered by said polydopamine to said polymer shell; and
incubating said human mesenchymal cells with said PEGylated polyhemoglobin nanocapsules.
